(19) United States
(12) Patent Application Publication
(10) Pub. No.: US 2007/0016204 A1
(43) Pub. Date: Jan. 18, 2007

(54) Spinal buttress device and method

(75) Inventors: Jaime Martinez, Pompton Plains, NJ (US); Alexandre M. DiNello, Cotuit, MA (US)

Correspondence Address: GIBBONS, DEL DEO, DOLAN, GRIFFINGER & VECCHIONE, 1 RIVERFRONT PLAZA, NEWARK, NJ, 07102-5497, US

(73) Assignee: Medical Device Concepts LLC.

(21) Appl. No.: 11/181,646

(22) Filed: Jul. 14, 2005

(51) Int. Cl. A61F 2/30

(52) U.S. Cl. 606/69

(57) ABSTRACT

A spinal buttress device for installation to the anterior of the spinal column. The device has a pair of opposed ends located adjacent the anterior of adjacent vertebrae with a flexible region therebetween. Either one or both of the ends of the device can be affixed to adjacent vertebrae or non-adjacent vertebrae and that affixation can be by screws passing through the device and into the vertebrae. The device is installed so as to span between the adjacent vertebrae and allow motion between those adjacent vertebrae. An example of the flexible region suitable for use in the device is a monolithic material having at least one slot formed therein. The dimensions of the slot, the cross section of the device and the material used to make the device, among other factors, enables the creation of a desired flexibility to the device. There is also a method of installing the device.

### FIELD OF THE INVENTION

The present invention relates to a device for positioning between adjacent vertebrae of a spinal column of a patient, and, more particularly, to a device that is affixed to one or more vertebrae and which has a desired flexibility to allow motion between the vertebrae while providing support for the spinal column.

### BACKGROUND OF THE INVENTION

In the field of spinal devices and techniques, there is a normal practice today of implanting artificial discs or motion devices intermediate to adjacent vertebrae as an alternative to the fusing of those vertebrae together. Since the fusion technique results in a loss of motion between the fused vertebrae, the use of an artificial disc is a procedure that allows the patient to have more flexibility in the spinal column so as to carry out more of the normal movement and function of the spinal column and restore the normal anatomical position of the spinal column.

There is a potential for the artificial disc replacement to migrate or extrude outwardly from the intradiscal space after implantation therein, and therefore, there can be a device that is affixed to the anterior of the spinal column that prevents such extrusion or migration of the artificial disc from the implanted position. One such device is shown and described in U.S. Publication 2003/0204260 A1 where the device includes a barrier plate that is affixed to a vertebra with a link member that allows some movement of the intradiscal device.

As such, there is a realization that with such procedures of installing artificial discs, there is a need for some device that may be emplaced and located along the anterior of the spinal column for a number of purposes. First, an anterior device can act as a buttress to prevent the potential migration of the artificial disc out of its emplaced position intermediate adjacent vertebrae. Secondly, an anterior device can act as a revision device that can be installed following a failed motion device surgery of an artificial disc or, thirdly, an anterior device can be emplaced as a stand alone device.

In any of the foregoing uses of an anterior device, it would be advantageous for the device to have certain inherent flexibility so that the movement of the anterior region of the vertebrae is allowed and it would also be advantageous to be able to design a particular desired flexibility into the anterior device in order to meet the needs of the spinal column or particular use of the anterior device.

Accordingly, it would be advantageous to have an anterior spinal buttress device for a spinal column that can be attached to the anterior of a spinal column so as to allow a desired flexibility between the vertebrae of that spinal column.

### SUMMARY OF THE INVENTION

Therefore, in accordance with the present invention there is an anterior spinal buttress device that is intended, for example, for the lumbar, thoracic and cervical sections of the spinal column and which provides a positive, yet flexible means of stabilizing the anterior of the spinal column. The device of this invention adds stability to the spinal column and also acts as an adhesion barrier or shield that prevents the growth of scar tissue.

The anterior spinal buttress device of the present invention includes a device that has opposed ends, that is, there are first and second ends, one of which is adapted to be attached to one of the adjacent vertebra and the other of which is adapted to be in close proximity to, and possible in direct contact with, the other adjacent vertebra of a spinal column. As alternate embodiment, the present spinal buttress device can span multiple vertebrae, that is, between vertebrae that are not adjacent to each other. In one embodiment, both of the first and second opposed ends are adapted to be attached to the adjacent vertebrae. In one described exemplary embodiment, the means of affixing either or both ends of the anterior spinal buttress device is by providing holes in both of the ends that accommodate bone screws that pass through the holes and the threads of the screws are screwed into the respective adjacent, or multiple vertebrae. Alternate means of affixation can include molybolts, wedges and the like.

Intermediate the first and second ends, there is a flexible region that allows the anterior spinal buttress device to flex in order to allow the adjacent vertebrae to also have a flexing action. The flexible region is specially constructed to be strong, monolithic device comprising, as an exemplary embodiment, a body having one or more slots formed therein in order to provide the necessary flexibility to the flexible region, and, of course, to the adjacent vertebrae.

For example, there may be single spiral slot or plurality of successive spiral slots formed in the flexible region of the anterior spinal buttress device in the manner as described in U.S. Pat. No. 5,488,761 of Leone, the disclosure of which is incorporated herein in its entirety by reference. As an alternate flexible region, the flexible region may comprise a body having alternating pairs of oppositely disposed slots formed therein with alternating pairs of slots being angularly offset, for example, at an angle of about 90 degrees as shown and described in co-pending patent application of Jaime Martinez, entitled “Flexible Shaft” and filed Jun. 3, 2005 as Ser. No. ______, the disclosure of which is hereby incorporated herein by reference in its entirety.

As a still further alternative, the flexible region may be constructed in accordance with the serpentine, helix-like slot forming the flexible member of U.S. Pat. No. 6,053,922 of Krause et al, and the disclosure of that patent is also incorporated herein in its entirety by reference.

The aforedescribed flexible region has the added advantage in that the degree of flexibility can be designed into the particular flexible region, that is, the flexibility of the region can be designed so as to have a desired flexibility by selecting among a number of parameters, such as, but not limited to, changing the spacing of the slots, selecting the material for making the region or changing the cross section of the body and any one or more of those selections can be made to design into the flexible region, the flexibility that is desired in the ultimate anterior spinal buttress device. Accordingly, the amount of flexibility of the anterior spinal buttress device of the present invention can be designed in accordance with the needs of the particular spinal column and/or use of the device.

The anterior spinal buttress device is also installed by means of a novel method. In particular, the ends of the device are both aligned and positioned so as to be proximate to or actually touching adjacent vertebrae and one of the ends of the device is attached to an adjacent vertebra by means of the bone screws passing through a hole or holes formed in that end and the threads screwed into the vertebra. As an alternate, both ends of the anterior spinal buttress device are attached to the adjacent vertebrae by the screws.

Thus, in the method, one of the ends of the anterior spinal buttress device is located abutting one of the adjacent vertebrae and firmly affixed thereto with the other end in close proximity to the adjacent vertebrae or in contact therewith. In a further embodiment, both of the opposed ends of the anterior spinal buttress device are affixed to the adjacent vertebrae with, in either embodiment, the flexible region between those ends providing the desired flexibility for the motion between the adjacent vertebrae.

Other features of the anterior spinal buttress device of the present invention and its method of installation will become more apparent in light of the following detailed description of a preferred embodiment thereof and as illustrated in the accompanying drawings.

### BRIEF DESCRIPTION OF THE DRAWINGS

FIG. 1 is a side view of an exemplary anterior spinal buttress device of the present invention affixed in position to a spinal column;

FIG. 2 is a front view of the device of FIG. 1 affixed to the spinal column;

FIG. 3 is a front view of an anterior spinal buttress device having an exemplary flexible region that is usable with the present invention;

FIG. 4 is a front view of an anterior spinal buttress device having another exemplary flexible region that is usable with the present invention;

FIG. 5 is a front view of an anterior spinal buttress device having a still further exemplary flexible region that is usable with the present invention;

### DETAILED DESCRIPTION OF THE INVENTION

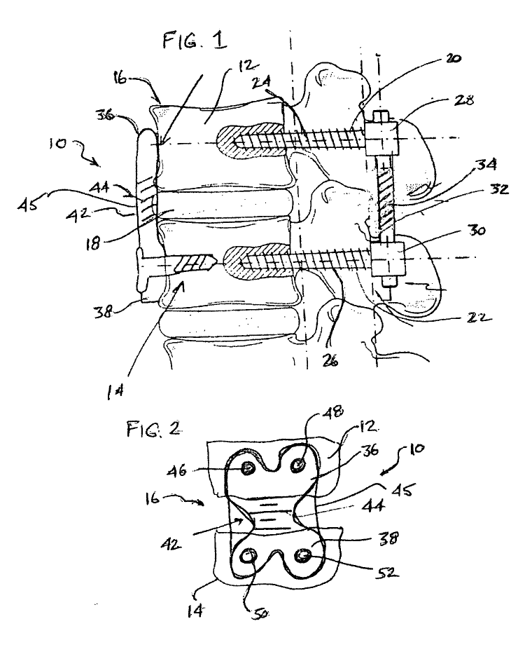

Referring now to FIG. 1, there is shown a side view of an exemplary anterior spinal buttress device 10 of the present invention affixed to the adjacent vertebra 12, 14 of spinal column 16. As can be seen, the vertebrae 12, 14 are separated by a disc 18 that may be a natural disc or may be a prosthetic device that has taken the place of a normal disc by a replacement thereof. As shown, the right side of the vertebrae 12, 14 as it appears in FIG. 1 is the posterior side facing outwardly of the patient and the left side of the vertebrae 12, 14 is the anterior side facing inwardly of the patient. Therefore it can be seen that the anterior spinal buttress device 10 is affixed proximate to the anterior of the spinal column 16.

On the posterior of the spinal column 16 there is a spinal stabilization system that is described more fully in co-pending U.S. patent application Ser. No. ______, filled ______ and entitled DYNAMIC SPINAL STABILIZATION SYSTEM AND METHOD and the disclosure of that patent application is hereby incorporated herein in its entirety by reference. In summary, however, the spinal stabilization system, for example, is affixed to the posterior of the spinal column 16 in order to allow some flexure of the adjacent vertebrae 12, 14 while stabilizing the spinal column. The spinal stabilization system includes a plurality of anchoring members 20, 22 that are affixed, respectively, to the vertebrae 12, 14. The anchoring members 20, 22 are identical and both include threads 24, 26 that are screwed into the vertebrae 12, 14 in order to solidly affix the anchoring members 20, 22 to the vertebrae 12, 14.

The anchoring members 20, 22 have external head ends 28, 30 extending from the threads 24, 26, respectively, and consequently also extending outwardly from the vertebrae 12, 14.

A flexible shaft 32 is affixed between the external head end 28 and 30 and is affixed thereto to span the adjacent vertebrae 12, 14.

As stated, the flexible shaft 32 may be specially constructed to be a strong, monolithic body having a slot or slots 34 formed therein in order to impart the flexibility to the otherwise relatively stiff shafts. The slot may be of the spiral type disclosed in Leone, U.S. Pat. No. 5,488,761, a serpentine helical slot as shown and described in Krause et al, U.S. Pat. No. 6,053,922 or may be of the type shown and described in copending U.S. patent application to Jaime Martinez, entitled “Flexible Shaft” having Ser. No. ______, filed Jun. 3, 2005, and the disclosure of the Leone, Krause et al patents and the Martinez application are hereby incorporated herein in their entirety by reference. That Martinez flexible shaft includes oppositely disposed slots in a body where alternating pairs of oppositely located slots are rotated 90 degrees about the outer periphery of the body such that the body can flex in multiple directions.

In any instance, the flexible shaft 32 can be designed for the desired flexibility by changing the configuration of the slot, the material of the body, the cross section of the body as well as other design changes so that the designer can determine the desired flexibility of the flexible shaft 32 depending upon the characteristics of the spinal column to which the flexible shaft 32 is installed.

Accordingly, while the spinal stabilization system is installed on the posterior side of the spinal column 16, the present invention, that is, the anterior spinal buttress device 10 is installed to the anterior side of the spinal column 16. As shown, the anterior spinal buttress device 10 includes opposed ends, that is, a first end 36 and a second end 38. As can be seen, the first and second ends 36, 38 abut up against the anterior of the adjacent vertebrae 12, 14 to effectively retain the disc 18 from any migration outwardly from its position between the adjacent vertebrae 12, 14. In the FIG. 1 embodiment, it can be seen that the first end 36 is merely abutting against the vertebra 12 while the second end 38 is actually affixed to the vertebra 24. As an alternative, the first end 36 need not actually abut against the vertebra 12 but may merely be in close proximity thereto.

The affixation of the second end 38 to the vertebra 24 can be carried out by a number of means, however, as shown, there is a bone screw 40 that passes through a suitable hole formed in the second end 38 and which is then screwed into the vertebra 14 to secure the second end 38 to the vertebra.

As such, only one end in the FIG. 1 exemplary embodiment, that is, the second end 38 is actually affixed to the vertebra 14 where the first end 36 is merely abutting or in close proximity to the vertebra 12. In alternate embodiments, of course, either the first or the second ends 36, 38 can be affixed to the vertebrae, 12, 14 respectively, or, as a further alternative, both of the first and second ends 36, 38 can be affixed, respectively, to the vertebrae 12, 14.

The body 42 of the anterior spinal buttress device 10 intermediate the first and second ends 36, 38 is formed so as to have a predetermined flexibility in the same manner, generally, as the flexible shaft 32 such that there is one or more slots 44 formed in the body 42 intermediate the first and second ends 36, 38 in order to provide that inherent and desired flexibility. There is thus formed a flexible region 45 intermediate the first and second ends 36, 38.

The flexibility of the body 42 is therefore specially designed and constructed to be of a predetermined amount by the formation of a strong, monolithic body having a slot or slots 44 formed therein in order to impart the flexibility to the otherwise relatively stiff shafts.

Again, as with the flexible shaft 32, the slots 44 may be of the spiral type disclosed in Leone, U.S. Pat. No. 5,488,761 or may be of the serpentine helical-like slot as shown and described in Krause et al, U.S. Pat. No. 6,053,922 or may be of the type shown and described in the aforementioned copending U.S. patent application to Jaime Martinez, entitled “Flexible Shaft”. Therefore, in order to obtain the desired characteristic flexibility, the body 42 can be configured of a particular cross section, can have the shape and/or depth of the slot or slots varied or the material can be selected to as to achieve whatever flexibility is desired in order to suit the particular needs of the patient and/or the particular use of the anterior spinal buttress device 10.

Turning to FIG. 2, taken along with FIG. 1, there is shown a front view of an exemplary anterior spinal buttress device 10 of the present invention affixed to the adjacent vertebrae 12, 14 and, as can be seen, there are a plurality of holes formed therein. Thus, there are two holes 46, 48 provided in the first end 36 and two holes 50, 52 formed in the second end 38 of the device and, as explained, the holes 46, 48, 50, and 52 are used to insert screws therethrough in order to affix those screws into the vertebrae 12, 14 to secure the anterior spinal buttress device 10 to the adjacent vertebrae 12, 14 of the spinal column 16.

Accordingly, as can now be appreciated, the anterior spinal buttress device 10 is installed to the anterior of the spinal column 16 by means of screws that are screwed into the adjacent vertebrae, whether to one vertebra or both vertebrae with the flexible region 45 therebetween so that there is a desired flexibility brought about by the design and construction of the device, so as to allow a flexure between the adjacent vertebrae along the anterior of the spinal column. In the embodiment illustrated in FIG. 2, the anterior spinal buttress device 10 has a relatively flat flexible region 45 that acts similar to a leaf spring to allow the desired flexibility between the first and second ends 36, 38. As will be seen, however, the flexible portion 45 can be constructed of a variety of means that can be produced with a controlled, predetermined flexibility between those first and second ends 36, 38 to provide the desired movement between the adjacent vertebrae.

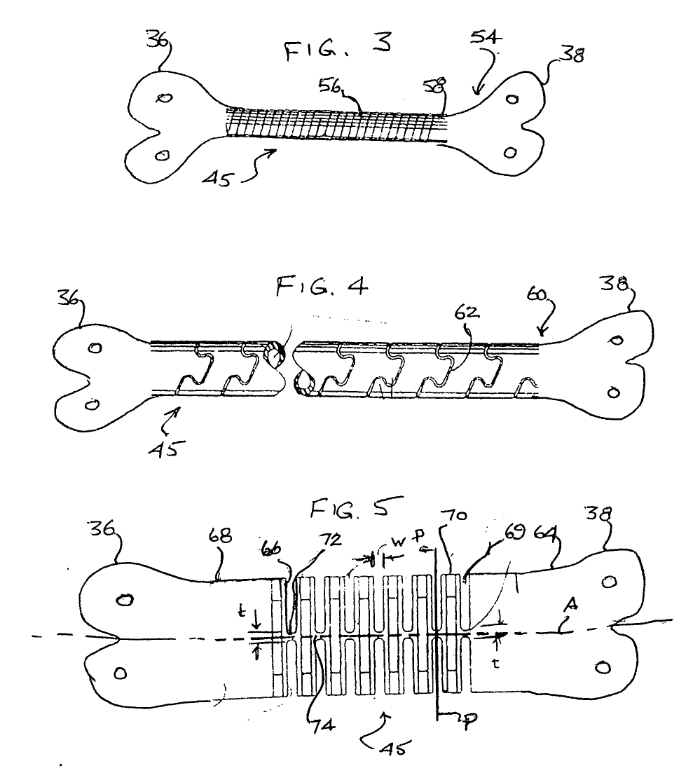

Turning now to FIG. 3, there is shown a front view of an exemplary anterior spinal buttress device 54 of the present invention. In FIG. 3, the anterior spinal buttress device 54 is constructed in accordance with U.S. Pat. No. 5,488,761 of Leone and the flexible region 45 located intermediate the first and second ends 36, 38 generally comprises a helical slot 56 that is formed into a shaft 58 and may have slot interruptions. The flexible region 45 provides some rotating or torsional give when rotary motion is along the flexible region 45 so that the flexible region 45 can have both flexibility along its longitudinal axis but also a small degree of rotational motion is allowed along that longitudinal axis. The helical slot 56 can be cut into the surface of the shaft 58 by means of continuously rotating the shaft 58 while providing relative motion of a cutting piece along the longitudinal length of the shaft. Thus, to adjust the pitch of the helical slots, the speed of the rotation of the shaft can be adjusted with respect to the relative longitudinal movement of the cutting tool or piece.

In FIG. 4, there is shown a front view of a further exemplary anterior spinal buttress device 60 of the present invention and where there is a specially formed serpentine, helical like slot 62 along the length of the flexible region 45 that is constructed in accordance with the disclosure of Krause et al U.S. Pat. No. 6,053,922.

Next, in FIG. 5, there is shown a top view of a still further anterior spinal buttress device 64 of the present invention. In this embodiment, there are a plurality of pairs of oppositely disposed slots 66 formed in a tubular body 68 and, as shown, those slots 66 are specially located and configured so as to create the desirable features of the present flexible shaft. The slots 66 are each comprised of an elongated opening 69 that is located along the peripheral outer surface 70 of the tubular body 68 and extend inwardly toward the longitudinal axis A of the tubular body 68. The elongated openings 69 of each pair of oppositely disposed slots 66 are located in a common plane, that is, at a right angle or 90 degrees to the longitudinal axis A of the tubular body 68 with the elongated openings 69 of each pair of slots 66 formed in the same plane orthogonal to the longitudinal axis A. The pairs of slots 66 extend inwardly such that each slot of a pair of slots 66 lies along the same plane P as the elongated openings 69, however, the slots 66 may be angled with respect to that plane or tapered inwardly such that while the elongated openings 69 of each pair of slots may be along the same lateral plane, the slots 66 themselves may be directed inwardly at an angle with respect to that plane.

The slots 66 are formed in the peripheral outer surface 70 of the tubular body 68 such that each slot 66 is less than 180 degrees about the peripheral outer surface 70 of the tubular body 68. Accordingly, since the pairs of slots 66 each are grouped in oppositely disposed slots 66, each slot is cut into the tubular body 68 and the slots 66 approach each other but terminate at ends 72 short of reaching the center of the tubular body 68, that is, the pairs of slots 66 are non-continuous and do not reach the longitudinal axis A as shown in FIG. 5.

Therefore, between each of the ends 72 of a pair of slots 66 there are formed web sections 74 that separate the ends 72 of the pairs of slots 66. Thus, each pair of oppositely disposed slots 66 as illustrated in FIG. 5 are in a common plane with the web sections 74 separating the ends 72 of each pair of slots that are formed in the tubular body 68 to approach each other but fall short of reaching the midpoint or longitudinal axis A of the tubular body 68. As such, the web sections 72 carry the rotational movement along the tubular body 68 while maintaining torque along that tubular body 68.

The pairs of slots 66 are alternately angularly oriented with respect to each other around the outer peripheral surface of the tubular body 68, that is, each succeeding pair of oppositely disposed slots 66 is rotated or displaced a predetermined angular amount from the orientation of the succeeding pair of slots 66. In the embodiment shown in FIG. 5, that displacement or rotation is about 90 degrees such that the slots 66 are formed in the tubular body every quarter of a turn. As such, there are at least a first and second pair of oppositely disposed slots 66 formed in the tubular body 68 with, for example, the first pair having one orientation and the next or second pair of slots 66 oriented 90 degrees rotated with respect to the first pair of slots 66 and so on throughout the tubular body 68.

While the angular displacement is illustrated in FIG. 5 to be 90 degrees, other angular displacements may be utilized and that angular displacement need not be the same or even consistent between successive pairs of slots 66.

The width w of the slots 66 can be predetermined in accordance with the desired flexibility of the completed anterior spinal buttress device 64, that is, the larger the width dimension w, the more flexible the flexible region 45 is and, consequently, also the eventual tubular body 68. The same is true of the depth of the slots 66 as the oppositely disposed slots approach each other nearing the midpoint or longitudinal axis A of the tubular body 68 i.e. the smaller the thickness t of the web sections 74 between the slots of each pair, the more flexible the flexible region 45 becomes. In one suitable embodiment, the thickness t of the web sections 74 is about the same, dimensionally, as the width w of the slots 66.

As can therefore be seen, the flexibility of the flexible region 45 can be different depending on the particular direction of flexing of the flexible region 45. One means of accomplishing that different flexibility would be to establish differing widths of pairs of slots 66 along two opposite sides of the tubular body 68 such that the flexibility in one direction of the pairs of slots 66 is different than the flexibility in another direction of motion, such as a direction at 90 degrees to the first direction. As such, the present anterior spinal buttress device 64 can be affixed to the anterior of the vertebrae of the patient in a particular orientation where the front to back flexibility of the spinal column can be different, and possibly more flexible, than the flexibility of the spinal column in a side to side direction.

The formation of the slots in this and other flexible regions can be accomplished by a variety of methods including milling the slots into the tubular body, using wire electrical discharge machining, water-jet machining, laser machining, spark erosion machining or rotary cutting machining. The material for the flexible regions and the anterior spinal buttress device itself can be any hard, rigid material including, but not limited to stainless steel, titanium, chrome cobalt molybdenum, polymers and carbon fiber composites.

While the present invention has been set forth in terms of a specific embodiment or embodiments, it will be understood that the anterior spinal buttress device and the method of installing the same disclosed herein may be modified or altered by those skilled in the art to other configurations. Accordingly, the invention is to be broadly construed and limited only by the scope and spirit of the claims appended hereto.

We claim:

1. A spinal buttress device comprising:
a device having first and second ends, the first and second ends adapted to rest in close proximity to the anterior portion of vertebrae of a patient, at least one of the first and second ends adapted to be attached to a vertebra,
said device having a flexible region disposed between the first and second ends so as to buttress material between said vertebrae while enabling desired relative motion of the vertebrae.

2. The spinal buttress device of claim 1 wherein the vertebrae are adjacent vertebrae.

3. The spinal buttress device of claim 1 wherein both said first and second ends are attached to vertebrae.

4. The spinal buttress device of claim 1 wherein flexible region has at least one slot formed therein to provide the flexibility of the flexible region.

5. The spinal buttress device of claim 4 wherein the at least one slot comprises a plurality of oppositely disposed pairs of slots offset at an angle with respect to an adjacent pair of slots.

6. The spinal buttress device of claim 4 wherein the adjacent slots are offset at an angle of about 90 degrees.

7. The spinal buttress device of claim 3 wherein the at least one slot is a serpentine helical-like slot formed therein along the linear length of the flexible region.

8. The spinal buttress device of claim 3 wherein the at least one slot is a spiral slot formed therein along the linear length of the flexible region.

9. The spinal buttress device of claim 1 wherein the flexible region comprises a flat spring member.

10. The spinal buttress device of claim 1 wherein the device includes at least one hole located at each of the first and second ends to carry out the attachment to a spinal vertebra by affixation devices.

11. The spinal buttress device of claim 10 wherein the affixation devices are screws that are passed through the holes and are screwed into the anterior of adjacent vertebrae.

12. A method for installing a spinal buttress device to the anterior portion of a spinal column, comprising the steps of:
providing a spinal buttress device having a first end, a second end and having a flexible region therebetween; and
attaching at least one of the first or second ends of the spinal buttress device to a vertebrae to buttress material between vertebrae while enabling desired relative motion of the vertebrae.

13. The method for installing a spinal buttress device as defined in claim 12 wherein the vertebrae are adjacent vertebrae.

14. The method for installing a spinal buttress device as defined in claim 12 wherein the step of attaching at least one of the first or second ends comprises attaching both the first and second ends of the spinal buttress device to adjacent vertebrae.

15. The method for installing a spinal buttress device as defined in claim 11 wherein the step of a attaching at least one of the first or second ends of the spinal buttress device comprises attaching the device by screwing screws into the vertebra.

16. The method for installing a spinal buttress device as defined in claim 11 wherein the step of providing a spinal buttress device having a flexible region comprises providing an anterior spinal buttress device having a flexible region with at least one spiral slot formed therein along its linear length.

17. The method for installing a spinal buttress device as defined in claim 11 wherein the step of providing a spinal buttress device having a flexible region comprises providing a spinal buttress device having a flexible region having at least two pairs of oppositely disposed slots formed therein with a pair of slots being angularly displaced with respect to an adjacent pair of slots.

18. The method for installing a spinal buttress device as defined in claim 14 wherein each adjacent pair of slots is displaced 90 degrees.

19. The method for installing a spinal buttress device as defined in claim 11 wherein the step of providing a spinal buttress device having a flexible region comprises providing an anterior spinal buttress device having a flexible region with at least one serpentine, helical-like slot.
